(19) United States
(12) Patent Application Publication
(10) Pub. No.: US 2019/0216894 A1
(43) Pub. Date: Jul. 18, 2019

(54) Medical Use of Creg Protein for Prevention or Treatment of Overweight, Obesity and Related Diseases Thereof

(71) Applicant: GENERAL HOSPITAL OF CHINESE PLA NORTHERN THEATER COMMAND, Shenyang, Liaoning (CN)

(72) Inventors: Yaling HAN, Shenyang, Liaoning (CN); Xiaoxiang TIAN, Shenyang, Liaoning (CN); Chenghui YAN, Shenyang, Liaoning (CN); Quanyu ZHANG, Shenyang, Liaoning (CN); Xiaolin ZHANG, Shenyang, Liaoning (CN); Dan LIU, Shenyang, Liaoning (CN)

(21) Appl. No.: 16/323,950

(22) PCT Filed: Jul. 26, 2017
(86) PCT No.: PCT/CN2017/094509

§ 371 Date: Feb. 7, 2019

(30) Foreign Application Priority Data
Aug. 8, 2016 (CN) 201610644971.9

(51) Int. Cl. A61K 38/17; A61P 3/04; A61P 3/08; A61P 3/06; A61P 1/16

(52) U.S. Cl. CPC A61K 38/1709; A61P 3/04; A61P 1/16; A61P 3/06; A61P 3/08

(57) ABSTRACT

Described is the use of an E1A activating gene repressor (CREG) protein, in particular to a use of a CREG protein or an active fragment thereof in manufacture of a medicament for prevention and/or treatment of overweight, obesity, insulin resistance, hyperlipidemia and related diseases. Also described is the use of a recombinant vector or recombinant cell expressing a CREG protein or an active fragment thereof in manufacture of a medicament for prevention and/or treatment of overweight, obesity, insulin resistance, hyperlipidemia and related diseases.

### TECHNICAL FIELD

The present invention relates to a medical use of Cellular Repressor of E1A-stimulated Genes (CREG), in particular to a pharmaceutical use of CREG protein or an active fragment thereof in manufacture of a medicament for prevention or treatment of overweight, obesity, insulin resistance, hyperlipidemia and related diseases thereof.

### BACKGROUND

Overweight and obesity are chronic metabolic diseases characterized by excessive accumulation or abnormal distribution of fat. According to the body weight index (BMI=weight/height²), the World Health Organization defines BMI≥25 Kg/m² as overweight, and BMI≥30 Kg/m² as obesity, while the domestic standard defines BMI≥24 Kg/m² as overweight, and BMI≥28 Kg/m² as obesity. If overweight is not controlled in time, it can develop into obesity. Overweight and obesity are both independent diseases, and can induce a variety of concomitant or secondary obesity-related diseases, including metabolic syndromes, pre-diabetes, type 2 diabetes, dyslipidemia, hypertension, nonalcoholic fatty liver diseases, polycystic ovary syndrome, sleep apnea, osteoarthritis, gastroesophageal reflux disease, etc., and thus are listed by WHO as one of the top ten risk factors for disease burden. In China, the obese population continues to grow. According to the “Investigation on Nutrition and Health of Chinese Residents” issued by the Ministry of Health in 2002, the overweight rate of adults in China is as high as 22.8% (200 million), and the obesity rate is 7.1% (60 million), which is doubled compared with that of 1980. It is estimated that by 2020, the obesity rate of China will be close to 10%. Therefore, prevention and control of obesity is an urgent task.

The continued increase in the incidence of overweight and obesity is due in part to changes in people's lifestyles and on the other hand to the lack of treatments for obesity. Medication is a powerful tool to control chronic metabolic diseases. At present, a variety of safe and effective drugs have been developed and can be chosen by clinicians for chronic metabolic diseases such as diabetes and hyperlipidemia. For overweight and obese patients, only five drugs approved by the US Food and Drug Administration (FDA), except for Orlistat, the other four have been approved in the past three years, and its long-term efficacy and side effects are not yet clear. In China, only one drug, i.e., Orlistat, is approved for the treatment of obesity. Orlistat is a lipase inhibitor that prevents the hydrolysis of triglycerides into absorbable free fatty acids and monoacylglycerols, which are not absorbed, thereby reducing caloric intake and controlling body weight. However, its effect on weight loss is not strong (in 1-4 years, drug-treated patients lost 2.5-3.2 kg of body weight compared with the placebo group), and it may cause some adverse reactions such as flatulence, oily spots and sense of stool emergency. In summary, there are currently few ideal safe and effective drugs for the treatment of overweight and obesity.

The mechanism of obesity is very complicated and is affected by many factors such as factors of heredity and environment. Studies have found that fat cell dysfunction, immune cell infiltration, and chronic low-level inflammatory response in adipose tissue are the major pathological changes in obesity. Among them, fat cell dysfunction is the initiating factor of obesity, but the key factors regulating the homeostasis of fat cells have not been elucidated.

CREG is a small molecule glycoprotein that is widely expressed in mature tissues and cells. CREG protein is mainly localized in the perinuclear Golgi and lysosomes, involved in the transport of lysosomal enzymes and the endocytosis of extracellular factors (Schahs P, Exp Cell Res, 2008, 314(16): 3036-3047; Kowalewski-Nimmerfall E, Biochim Biophys Acta, 2014, 1843(12): 2900-2912). Moreover, a large number of studies have shown that CREG protein is involved in the occurrence and progression of various cardiovascular diseases such as hypertension, vascular remodeling, atherosclerosis, myocardial ischemia-reperfusion injury, myocardial infarction, etc., and is an important factor in maintenance of cardiovascular homeostasis and embryonic development. However, the role of CREG protein in obesity and its related diseases as well as its mechanism are still unclear.

### SUMMARY

The inventors of the present invention have found through a large number of experiments that the expression of CREG in adipose tissue of obese mice is significantly lowered. Exogenous supplementation of CREG protein into obese mice can significantly reduce body weight, reduce adipose tissue weight, reduce liver tissue steatosis, lower blood lipid level, improve secretion of adipokines, and improve insulin resistance. The above results indicate that exogenous supplementation of CREG protein can be used for prophylaxis or treatment of overweight, obesity, insulin resistance, hyperlipidemia and related diseases. The present invention has been completed based on the above findings.

A first aspect of the invention relates to the use of a CREG protein or an active fragment thereof in manufacture of a medicament for prophylaxis and/or treatment of obesity.

The present invention also relates to the use of a nucleic acid molecule encoding a CREG protein or an active fragment thereof, a recombinant vector or a recombinant cell expressing a CREG protein or an active fragment thereof, in manufacture of a medicament for prophylaxis and/or treatment of overweight, obesity, insulin resistance, hyperlipidemia and related diseases thereof.

In an embodiment of the invention, the recombinant vector comprises a nucleic acid molecule encoding a CREG protein or an active fragment thereof.

The invention also relates to the use of an agent for detecting an expression level of a CREG protein or an active fragment thereof in manufacture of a kit, wherein the kit is useful in predication and/or evaluation of therapeutic effect and prognosis of overweight, obesity, insulin resistance, hyperlipidemia.

The invention also relates to a use of a CREG protein or an active fragment thereof in screening for a medicament for prophylaxis and/or treatment of overweight, obesity, insulin resistance, hyperlipidemia.

In an embodiment of the present invention, the CREG protein or an active fragment thereof can be used as a target protein in screening for a medicament for prophylaxis and/or treatment of overweight, obesity, insulin resistance, hyperlipidemia and related diseases; for example, an agent capable of promoting the up-regulation of expression of CREG protein or an active fragment can be used as a drug for prophylaxis and/or treatment of overweight, obesity, insulin resistance, hyperlipidemia, and related diseases.

The invention also relates to a composition, which comprises a CREG protein or an active fragment thereof, a nucleic acid molecule encoding a CREG protein or an active fragment thereof, a recombinant vector or recombinant cell expressing a CREG protein or an active fragment thereof, and optionally a pharmaceutically acceptable carrier or excipient, the composition can be used for prophylaxis and/or treatment of overweight, obesity, insulin resistance, hyperlipidemia and related diseases.

The invention also relates to a kit, which comprises an agent for detecting the expression level of a CREG protein or an active fragment thereof, and the kit can be used for prediction and/or evaluation of therapeutic effects and prognosis of overweight, obesity, insulin resistance, hyperlipidemia and related diseases.

In the present invention, the CREG protein is a recombinant CREG protein derived from a mammal, particularly from a human. In a preferred embodiment of the invention, the GenBank number of the CREG protein is NP_003842.1 (SEQ ID NO: 1). In a preferred embodiment of the invention, the GenBank number of the CREG gene is NM_003851.2 (SEQ ID NO: 2).

In the present invention, the active fragment of the CREG protein refers to a fragment having the function of the CREG protein, which may be a part of the CREG protein, or a fragment obtained by deleting, adding or replacing an amino acid sequence of the CREG protein; the methods for obtaining the active fragments of the CREG protein are known in the art, for example, the active fragment is a fragment comprising a portion of the CREG protein that binds to a ligand or receptor, or a fragment that retains the function of the CREG protein after deletion, addition or substitution of an amino acid. It is well known to those skilled in the art that some key amino acids on the CREG protein are closely related to the activities, and the mutation thereof may affect the activities of the protein, for example, the mutations of lysine at positions 136 and 137 of the CREG protein to alanine, or the mutations of amino acid deletion at positions 141-144 of CREG protein would affect the activity and function of the protein (Sacher M, PNAS, 2005; 102 (51): 18326-18331). Those skilled in the art can avoid the above-mentioned sites which may affect the activity as needed, and perform alteration, addition or substitution on other sites, so that the modified CREG protein still has the activity or function of the CREG protein.

In the present invention, the overweight and obesity have well-known meanings in the art, with or without obesity-related diseases.

In the present invention, the overweight or obesity-related disease has a meaning well-known in the art, and refers to a series of diseases accompanying or secondary to being overweight or obese, including metabolic syndrome, pre-diabetes, type 2 diabetes, and dyslipidemia.

In the present invention, the prevention and/or treatment of overweight or obesity means inhibiting or slowing the occurrence of overweight or obesity, inhibiting or slowing the occurrence of an overweight or obesity-related disease.

In the present invention, the use of detection of the expression level of the CREG protein or an active fragment thereof in prediction and/or evaluation refers to that when the expression level of the CREG protein or an active fragment thereof in blood, tissue or cells is lower than a reference value, the prediction of occurrence of overweight or obesity, or evaluation of therapeutic effect or prognosis thereof can be carried out.

In the present invention, the mammal may be, for example, a rat, a mouse, a dog, a miniature pig, a monkey, a human or the like.

In the present invention, the expression level of the CREG protein or an active fragment thereof can be detected by a method known in the art, for example, the expression level of the CREG protein can be detected by amplification of CREG mRNA by polymerase chain reaction and quantitative reaction, or by Western Blot.

In the present invention, the expression level of protein refers to the level of mRNA or the level of protein.

In the present invention, the up-regulation/down-regulation of expression of a protein in a tissue/cell refers to an increase or decrease by at least 20%, 30%, 40%, 50%, 60%, 70%, 80%, 90%, 100%, or an increase by more than 100%, of the protein level or mRNA level in the tissue/cell. The up- or down-regulation described therein refers to comparison to an uninterrupted tissue/cell (e.g., a tissue/cell transfected with control vector).

### BRIEF DESCRIPTION OF THE DRAWINGS

FIGS. 1A-D show the preparation of an obesity model of high-fat fed C57BL/6J mice and the detection of CREG protein expression in adipose tissue. FIG. 1A shows that after C57BL/6J mice were high-fat fed for 16 weeks, significant obesity occurred. FIG. 1B shows body weights of C57BL/6J mice at different weeks after being high-fat fed and normal fed. *p<0.05, **p<0.01, ***p<0.001 (compared to the normal feeding group). FIG. 1C shows the expression of CREG protein in adipose tissues of the high-fat fed obesity mice and the control mice detected by immunohistochemical staining method. FIG. 1D shows the expression of CREG protein in adipose tissues of the high-fat fed obesity mice and the control mice detected by Western Blot method. **p<0.01 (compared to the normal feeding group).

FIGS. 2A-G show that the CREG protein treatment significantly alleviated obesity in high-fat fed C57BL/6J mice. FIG. 1A shows that when the high-fat fed C57BL/6J mice were treated with CREG protein (150 μg/kg·d), CREG protein treatment significantly alleviated obesity in the mice. FIG. 2B shows the results of body weights at different weeks in the mice of 3 groups, i.e., normal feeding, high-fat feeding and high-fat feeding+CREG protein treatment groups, suggest that CREG protein treatment can significantly alleviate the body weight of obesity mice. *p<0.05, **p<0.01 (compared to the high-fat feeding group). FIG. 2C shows the results of food intake doses at different weeks in the mice of 3 groups, i.e., normal feeding, high-fat feeding and high-fat feeding+CREG protein treatment groups, suggest that CREG protein does not affect the caloric intake of mice. FIG. 2D shows the results of HE staining of epididymal adipose tissue at time point of 16 weeks in the mice of 3 groups, i.e., normal feeding, high-fat feeding and high-fat feeding+CREG protein treatment groups, suggest that CREG protein significantly reduced the size of fat cells. FIG. 2E shows a statistical analysis of the adipose cell area of epididymal adipose tissue at time point of 16 weeks in the mice of 3 groups, i.e., normal feeding, high-fat feeding and high-fat feeding+CREG protein treatment groups. **p<0.01 (compared to the high-fat feeding group). FIG. 2F shows the weights of inguinal and epididymal white adipose tissue at time point of 16 weeks in the mice of 3 groups, i.e., normal feeding, high-fat feeding and high-fat feeding+CREG protein treatment groups, suggest that CREG treatment significantly reduced the weight of white fat in obesity mice. **p<0.01 (compared to the high-fat feeding group). FIG. 2G shows the results of CREG immunohistochemical staining of epididymal adipose tissue at time point of 16 weeks in the mice of 3 groups, i.e., normal feeding, high-fat feeding and high-fat feeding+CREG protein treatment groups, suggest that the amount of CREG in adipose tissue could be increased after CREG protein treatment.

FIGS. 3A-C show that the CREG protein treatment significantly alleviated liver steatosis in high-fat fed C57BL/6J mice. FIG. 3A shows the results of HE staining of liver tissue at time point of 16 weeks in the mice of 3 groups, i.e., normal feeding, high-fat feeding and high-fat feeding+CREG protein treatment groups, suggest that the CREG treatment could significantly alleviate liver steatosis in obesity mice. FIG. 3B shows the results of liver weight at time point of 16 weeks in the mice of 3 groups, i.e., normal feeding, high-fat feeding and high-fat feeding+CREG protein treatment groups. *p<0.05 (compared to the high fat fed group). FIG. 3C shows the results of CREG protein immunohistochemical staining of liver tissue at time point of 16 weeks in the mice of 3 groups, i.e., normal feeding, high-fat feeding and high-fat feeding+CREG protein treatment groups, suggest that the CREG protein treatment could increase the amount of CREG protein in liver tissue of obesity mice.

FIGS. 4A-C show that the CREG protein treatment reduced the blood lipid level and improved the expression of adipokines in obesity mice. FIG. 4A shows the results of blood lipid level at time point of 16 weeks in the mice of 3 groups, i.e., normal feeding, high-fat feeding and high-fat feeding+CREG protein treatment groups, suggest that the CREG protein treatment could significantly reduce the levels of total cholesterol, triglycerides and low-density lipoprotein cholesterol in the obesity mice. *p<0.05 (compared to the high-fat feeding group). FIGS. 4 B and C show the results of adipokine leptin and adiponectin levels at time point of 16 weeks in the mice of 3 groups, i.e., normal feeding, high-fat feeding and high-fat feeding+CREG protein treatment groups, suggest that the CREG protein treatment could inhibit the expression of leptin in obesity mice and promote the expression of adiponectin in obesity mice.

FIG. 5 shows that the CREG protein treatment improved insulin resistance in the obese mice. The results of glucose tolerance test and insulin tolerance test performed at time point of 16 weeks in the mice of 3 groups, i.e., normal feeding, high-fat feeding and high-fat feeding+CREG protein treatment groups, suggest that the CREG protein treatment could significantly improve insulin resistance in obesity mice.

### DETAILED DESCRIPTION

The embodiments of the present invention will be described in detail below in conjunction with examples, however, the following examples are intended to illustrate the invention and are not intended to limit the scope of the invention. In the examples, those are not specified with specific conditions are carried out according to the conventional conditions or the conditions recommended by the manufacturers. The reagents or instruments used that are not indicated by the manufacturer are conventional products that are commercially available.

The experimental data of the present invention are all percentages. The chi-square test was used for comparison of two-sample rates, and the statistical processing was performed using the SPSS 19.0 software package. There was a statistical difference at P<0.05.

### Example 1: Preparation of Obesity Model of High-Fat Fed C57BL/6J Mice and Detection of CREG Protein Expression in Adipose Tissue

Establishment of obesity model of C57BL/6J mice: 40 male 8-week-old C57BL/6 mice [body weight (22.3±1.2) g] were divided into the following two groups by random table method: normal feeding group and high fat feeding group, 20 mice in each group. The normal feeding group was fed with normal diet feed (3.85 kcal/g, 10% calories were provided by fat, American Research Diet Company), and the high fat feeding group was fed with high fat diet (5.24 kcal/g, 60% calories were provided by fat, American Research Diet Company). After being continuously fed for 16 weeks, the mice were fed with standard conditions and received 12 hours of light without restriction on diet and water intake.

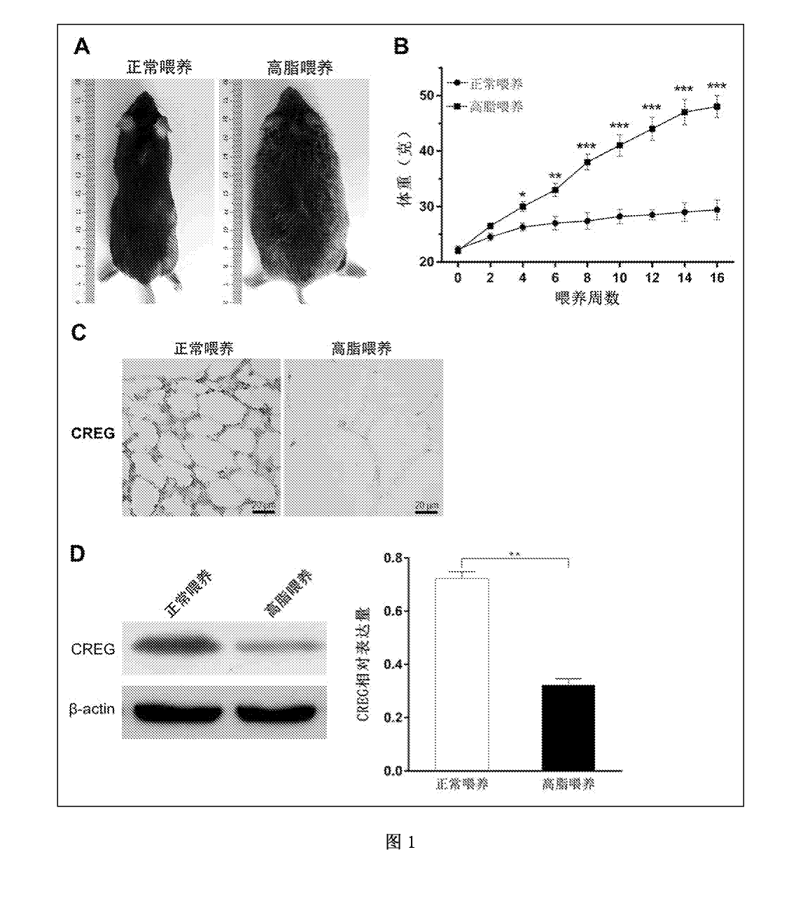

The results showed that after 16 weeks of feeding, the general morphology of the mice was observed, and it was found that the mice in the high-fat feeding group showed significant obesity, indicating that the obesity model was established successfully (FIG. 1A).

Body weight results of the high-fat feeding and normal feeding C57BL/6J mice at different weeks: The mice of each of the groups were weighed every 2 weeks. Fasting and water deprivation were carried out for 12 h before weighing. Each mouse was weighed 3 times, recorded and averaged.

The results showed that the body weight of the C57BL/6J mice of the high-fat feeding group was significantly higher than that of the normal feeding control group at the 4^{th} week of feeding. Subsequently, with the increase of feeding time, the weight gain of the mice in the high-fat feeding group was more significant, and the body weight was close to 50 g at the 16^{th} week, while that of the control group was only about 30 g. The above results indicate that we successfully established the mice model of obesity induced by high-fat diet (FIG. 1B).

The expression of CREG protein was detected by immunohistochemical staining method in adipose tissues of high-fat fed obesity mice and control mice:

### Methods

1) When feeding for 16 weeks, the white fat of epididymis tissue was taken and fixed with 4% paraformaldehyde, embedded in paraffin, and sliced at 5 μm;

2) the slices were conventionally dewaxed with xylene, washed with various stages of ethanol to water: xylene (I) for 5 minutes→xylene (II) for 5 minutes→100% ethanol for 2 minutes→95% ethanol for 1 minute→80% ethanol for 1 minute→75% ethanol for 1 minute→distilled water for 2 minutes;

3) the paraffin slices were immersed into an antigenic repair solution of appropriate pH value, boiled at 100° C. for 40 min, and naturally cooled to room temperature;

4) to each of the slices was added one drop of 3% hydrogen peroxide followed by incubation for 10 min at room temperature to eliminate endogenous peroxidase activity; the slices were rinsed with PBS 3 times, 5 min each time;

5) the slices were blocked with 10% goat serum and incubated for 10 min at room temperature;

6) the serum was discarded, a 1:100 dilution of anti-CREG (American Abcam) antibody was added, followed by standing overnight at 4° C., rewarming at room temperature for 30 min the next day, and rinsing with PBS 3 times, 5 min each time;

7) the PBS was discarded, a biotin-labeled secondary antibody was added, incubated for 30 min at room temperature, and rinsed 3 times with PBS, 5 min each time;

8) the PBS was discarded, a streptomyces avidin-peroxidase solution was added, followed by incubating for 10 min at room temperature, and rinsing 3 times with PBS, 5 min each time;

9) the PBS was discarded, a DAB solution was added and incubated for 5-10 min at room temperature, and the color development was stopped according to the situation; and

10) the slices were washed with tap water thoroughly, counterstained with hematoxylin, and sealed.

The results showed that the volume of adipocytes in the white adipose tissue of the epididymis of the mice of the high-fat feeding group was significantly increased, and the expression of CREG was significantly lower than that of the normal feeding group, suggesting a negative correlation between CREG expression and obesity (FIG. 1C).

The expression of CREG protein was detected by Western Blot method in adipose tissue of high-fat fed obesity mice and control mice: To detect the expression of CREG in adipose tissue of mice, white adipose tissue was extracted from the epididymis of the mice of the high-fat feeding group and the normal feeding group respectively after being fed for 16 weeks, and the expression of CREG protein in the two groups was detected by the Western Blot method. The extracted tissue was first weigh, then a protein lysate was added in a ratio of 1 mg: 10 μL and placed on ice for 20 min. The protein supernatant was collected by centrifugation at 13,000 rpm at 4° C. The protein concentration in the lysate was determined using a BCA colorimetric kit. After being boiled at 95° C. for 5 min, 50 μg of protein was subjected to SDS-PAGE electrophoresis with 12% separation gel, and the electrophoresis termination time was determined. The sample was transferred to a PVDF membrane at a voltage of 21 V for 45 min; it was blocked at room temperature for 1.5 h in a TBS-T diluted 5% skim milk powder and then incubated at 4° C. overnight. 1:1000 anti-CREG (Abcam, USA) antibody, 1:1000 anti-beta-actin (Santa cruz, USA) antibody were separately used as primary antibody, horseradish peroxidase-labeled goat anti-mouse antibody (Cell Signalling, USA) was used as secondary antibody, Western Blot detection was performed, and ECL kit (Amersham, USA) was used for luminescence developing. The CREG antibody and beta-actin antibody were used to detect expression bands of proteins of about 24 KD and 43 KD, respectively. The grayscale measurement of the bands was performed using Quantity One software and statistical analysis was performed.

The results showed that the expression of CREG protein in adipose tissue was significantly lower in the high-fat fed obesity mice than that in the control mice (FIG. 1D), suggesting that the CREG expression in adipose tissue is negatively correlated with obesity, and CREG may have a role of fighting obesity.

### Example 2: CREG Protein Treatment Significantly Alleviated Obesity in High-Fat Fed C57BL/6J Mice

CREG protein treatment could alleviate obesity caused by high fat feeding in mice: 60 male 8-week-old C57BL/6 mice were divided into the following three groups by random table method: normal feeding group, high fat feeding group, high fat feeding+CREG protein treatment group, 20 mice in each group. The normal feeding and high fat feeding methods were the same as in Example 1. In the high fat feeding+CREG protein treatment group, during the high-fat feeding, the mice were administrated with CREG protein (Abcam, USA; 150 μg/kg·d) by subcutaneously embedded micro-osmotic pump (Alzet, USA), and the treatment was carried out for 16 weeks. The mice were then treated with standard feeding conditions and received light for 12 h, without restriction in diet and water intake. The general morphology of the mice was observed after 16 weeks.

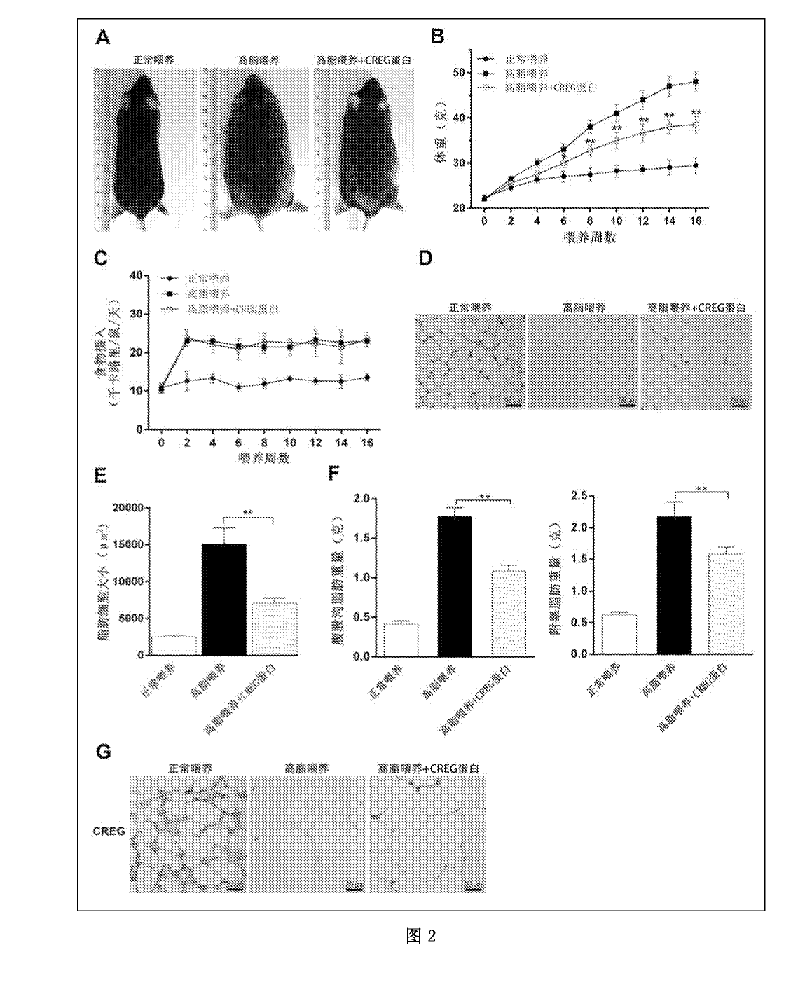

The results showed that the high-fat fed mice exhibited significant obesity, while the obesity of mice was significantly alleviated after treatment with CREG protein, indicating that CREG has the effect of treating obesity FIG. 2A).

Comparison of body weight of the three groups of mice: Each group of mice was weighed every two weeks, with fasting and water deprivation for 12 hours before weighing. Each mouse was weighed 3 times, recorded and averaged.

The results showed that the CREG protein treatment group showed a significant decrease in the body weight in comparison with the high fat-fed group, indicating that CREG has the effect of treating obesity (FIG. 2B).

Comparison of caloric intake of the three groups of mice: The quality of total feed for each mouse in 2 weeks was weighed, in which the caloric intake of the normal feeding group was calculated at 3.85 kcal/g of normal feed, the caloric intakes of the high fat feeding group and the CREG protein treatment group were calculated at 5.24 kcal/g of high fat feed, and the average daily caloric intake per mouse was obtained by dividing the quality by 14.

The results showed that the high-fat feeding group and the high-fat feeding+CREG protein treatment group all had high-fat diet, so their caloric intakes were significantly higher than that of the normal feeding control group. However, there was no difference in caloric intake between the high-fat feeding group and the high-fat feeding+CREG protein treatment group, indicating that CREG protein treatment did not affect appetite and caloric intake in mice (FIG. 2C).

Comparison of adipocyte size and adipose tissue weight in the three groups of mice: The white adipose tissues of epididymis were taken for HE staining analysis and treated as follows.

1) The white adipose tissues of epididymis were taken and fixed with 10% formaldehyde, conventionally embedded in paraffin, and sliced at 5 μm;

2) the slices were conventionally dewaxed with xylene, washed with various stages of ethanol to water: xylene (I) for 5 min→xylene (II) for 5 min→100% ethanol for 2 min→95% ethanol for 1 min→80% ethanol for 1 min→75% ethanol for 1 min→distilled water for 2 min;

3) the slices were stained with hematoxylin for 5 min, and rinsed with tap water;

4) the slices were differentiated with hydrochloric acid in ethanol for 30 s;

5) the slices were immersed in tap water for 15 min;

6) the slices were placed in eosin solution for 2 min;

7) the slices were conventionally dehydrated to be transparent, and sealed: 95% ethanol for 1 min→95% ethanol for 1 min→100% ethanol (I) for 1 min→100% ethanol (II) for 1 min→xylene (I) for 1 min→xylene (II) for 1 min→sealed with neutral resin; and

8) the morphology was observed under a microscope and stored for statistical analysis.

In addition, the inguinal and epididymal white adipose tissues of each group of mice were weighed and statistically analyzed.

The results showed that compared with the high-fat feeding group, the size of adipocytes in the CREG treatment group was significantly reduced (FIGS. 2D and 2E), and the weights of the inguinal and epididymal white adipose tissues significantly decreased (FIG. 2F), indicating that the CREG protein has the effect of treating obesity.

CREG immunohistochemical staining of epididymal white adipose tissue in the three groups of mice: The epididymal white adipose tissues of the three groups of mice were taken and subjected to CREG immunohistochemical staining. The specific method was the same as in Example 1.

The results showed that compared with the high-fat feeding group, the CREG staining of adipose tissues was significantly enhanced after the CREG protein treatment (FIG. 2G), indicating an increase of CREG protein amount, suggesting that CREG protein may reverse the obesity caused by high-fat feeding by increasing the CREG protein expression in adipose tissues.

### Example 3: CREG Protein Treatment Significantly Alleviated Steatosis of Liver in High-Fat Fed C57BL/6J Mice

HE staining of liver tissues in the three groups of mice: At the 16^{th} week of feeding, the liver tissues of each group of mice were taken for HE staining, and the specific method was the same as in Example 2.

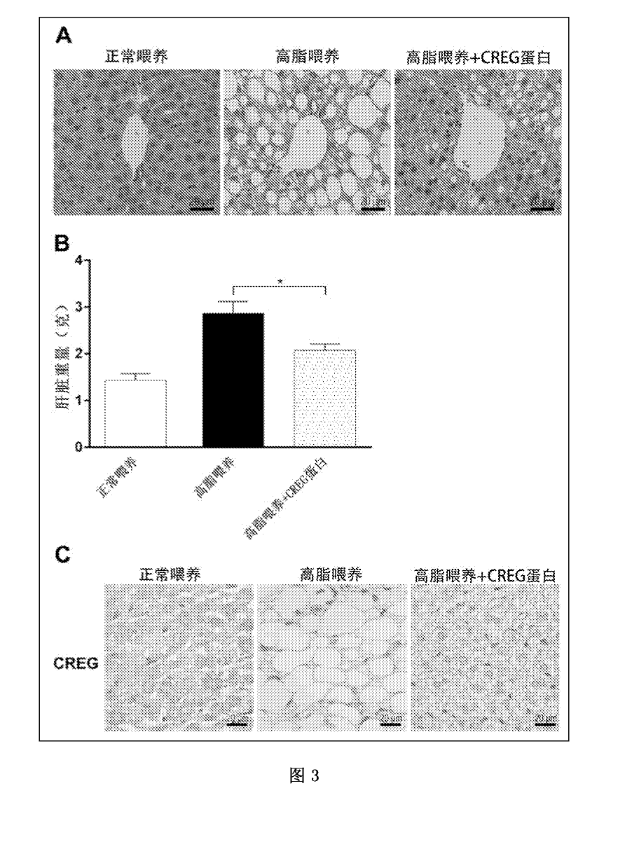

The results showed that compared with the normal feeding control mice, the liver of the mice after the high-fat feeding showed significant steatosis, and the liver cells were occupied by a large number of lipid droplets. Compared with the mice of the high-fat feeding group, the degree of hepatic steatosis was significantly reduced after treatment with CREG protein in the high-fat fed mice (FIG. 3A), indicating that CREG treatment could combat obesity-related fatty liver in mice.

Comparison of liver tissue weights of the three groups of mice: At the 16^{th} weeks of feeding, the mice were sacrificed and their liver tissue were taken. After being washed with PBS, the residual liquid was removed by filter paper as much as possible. The liver of each mouse was weighed 3 times and averaged, recorded and statistically analyzed.

The results showed that compared with the normal feeding control group, the liver weight increased significantly in the mice after high fat feeding. Compared with the mice of the high-fat feeding group, the liver weight of the mice of the high-fat feeding+CREG protein treatment group was significantly reduced (FIG. 3B), indicating that the CREG protein treatment could alleviate the abnormal increase in liver weight in the obese mice.

CREG protein immunohistochemical staining of liver tissues of the three groups of mice: The liver tissues of the three groups of mice were taken at the 16^{th} weeks, and subjected to CREG protein immunohistochemical staining, in which the specific method was the same as in Example 1.

The results showed that compared with the high-fat feeding group, the CREG staining of liver tissue was significantly enhanced after the CREG protein treatment in the high-fat fed mice (FIG. 3C), indicating an increase of CREG protein, suggesting that exogenous CREG protein may combat liver steatosis in obesity mice by increasing CREG protein expression in liver tissue.

### Example 4: CREG Protein Treatment can Reduce Blood Lipid Levels and Improve Expression of Adipokines in Obesity Mice

Measurement of blood lipid levels in the three groups of mice: At the 16^{th} week of feeding, 1-2 ml of arterial blood was collected from of each group of mice by carotid artery blood sampling method. The serum was collected after centrifugation at 3000 rpm, a portion thereof was stored in a cryogenic refrigerator for subsequent measurement, and another portion was used for measurement of blood lipid level. The blood lipid testing was performed using a Hitachi automatic biochemical analyzer.

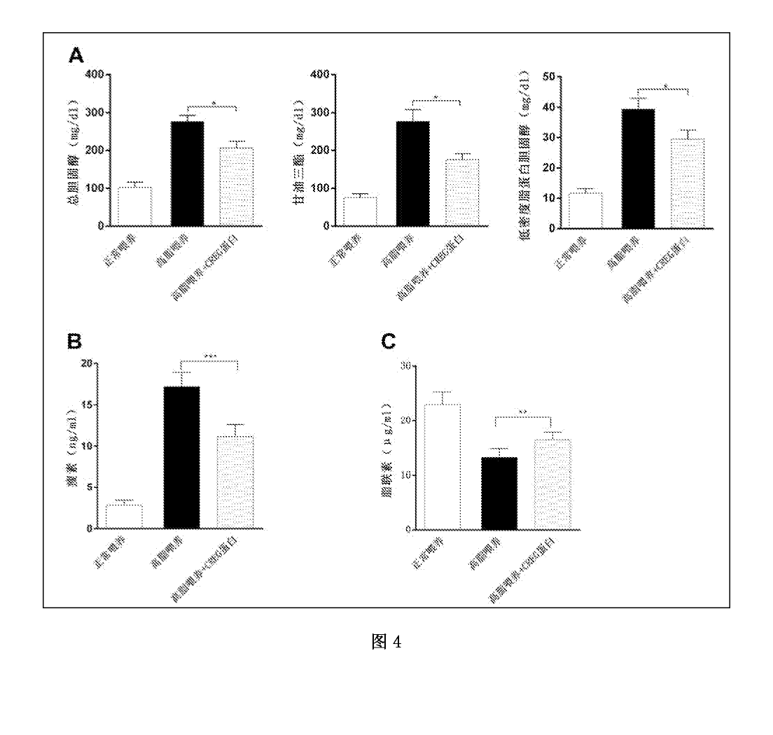

The results showed that compared with the normal feeding group, the plasma total cholesterol, triglyceride and low-density lipoprotein cholesterol were significantly increased in the mice after high-fat feeding, indicating that obesity occurred and accompanied by obvious dyslipidemia. After administration of CREG protein in the mice, the plasma total cholesterol, triglyceride and low-density lipoprotein cholesterol levels were significantly lowered in comparison with those of the high-fat feeding group (the results are shown in FIG. 4A), indicating that CREG protein has therapeutic effect of treating hyperlipidemia associated with obesity.

Determination of adipokine leptin and adiponectin levels in the three groups of mice: The plasma samples were obtained in the same manner as in Example 1, and used for the determination of leptin and adiponectin levels. The determination was performed by ELISA method, and the leptin and adiponectin ELISA assay kits were purchased from RD Corporation of the United States of America. The method was as follows:

1) Samples and standards were prepared, microplate strips were taken out, and 50 μl of Diluent Buffer was added to each well;

2) 50 μl of standard, control and sample was separately added to each well, and 2 duplicate wells were set for each sample, wells were covered with adhesive patch, and gently tapped for 1 min to mix evenly;

3) strips were incubated for 2 h at room temperature. The liquid was discarded, each well was washed with 400 μl of Wash Buffer 5 times. The liquid was removed as much as possible in every step. All remaining Wash Buffer was removed as much as possible in the final washing step;

4) 100 μl of Mouse leptin/Adiponectin Conjugate was added to each well, a new adhesive patch was used for changing, and incubation was performed for 2 h at room temperature;

5) the plate was washed 5 times. 100 μl of Substrate Solution was added to each well and incubation was performed for 30 min at room temperature in the dark;

6) 100 μl of Stop Solution was added to each well and the plate was gently tapped to mix evenly;

7) the results were read by a microplate reader within 30 min, i.e., read at 450 nm wavelength, and corrected at 540 nm or 570 nm, 3 data points were read in each experiment and each experiment was repeated 3 times;

8) a standard curve was plotted and the levels of leptin and adiponectin in each group of samples were calculated accordingly, and a statistical analysis was carried out.

The results showed that compared with the normal feeding group, the level of harmful adipokine leptin in plasma was significantly increased after high-fat feeding, while the level of beneficial adipokine adiponectin was significantly decreased. After exogenous administration of CREG protein in the high-fat fed mice, the leptin level was significantly decreased in comparison with the high-fat feeding group, while the adiponectin level was significantly elevated (FIGS. 4B and 4C), indicating CREG Protein can improve the expression of adipokines associated with obesity in mice.

### Example 5: CREG Protein Treatment can Improve Insulin Resistance in Obesity Mice

Glucose tolerance test: The mice were intraperitoneally injected with a total volume of 0.1 ml of glucose aqueous solution at a dose of 2 g/kg, and the blood glucose levels of tail vein blood were measured at 15 min, 30 min, 45 min, 60 min and 120 min after injection to determine the function of islet cells.

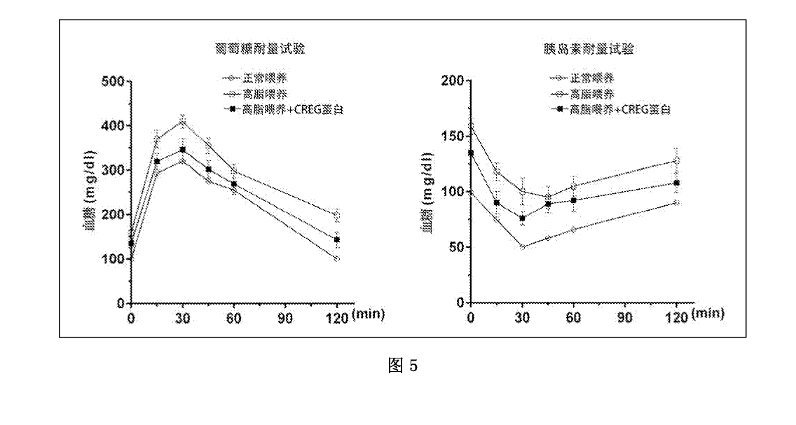

The results showed that compared with the normal feeding group, the blood glucose level of the high fat feeding group was significantly increased at the same time points, suggesting that the glucose tolerance was impaired after high fat feeding. After exogenous administration of CREG protein in the high-fat fed mice, the blood glucose level was significantly lowered in the high-fat fed mice at the same time points (FIG. 5), indicating that CREG protein could improve the impaired glucose tolerance accompanied with obesity.

Insulin tolerance test: The mice were injected intraperitoneally with human insulin at a dose of 0.75 U/kg, and the blood glucose levels of tail vein blood were measured at 15 min, 30 min, 45 min, 60 min and 120 min after injection to determine insulin resistance.

The results showed that compared with the normal feeding group, the blood glucose level of the high fat feeding group was significantly increased at the same time points, suggesting that insulin resistance occurred in the mice after high fat feeding. After exogenous administration of CREG protein in the high-fat fed mice, the blood glucose level was significantly lowered in the high-fat fed mice at the same time points (FIG. 5), indicating that CREG protein can improve insulin resistance associated with obesity in mice.

The above results suggest that CREG protein is expected to be an effective drug for the prevention and treatment of overweight, obesity, insulin resistance, hyperlipidemia and related diseases thereof.

Although the embodiments of the invention has been described in detail, it will be understood by those skilled in the art that various modifications and alterations of the details are possible in light of the teachings of the invention. The full scope of the invention is given by the appended claims and any equivalents thereof.

1. (canceled)

2. (canceled)

3. A method of evaluating therapeutic effect and prognosis of overweight, obesity, insulin resistance, or hyperlipidemia in blood, tissue or cells, comprising detecting an expression level of a CREG protein or an active fragment thereof or a nucleic acid molecule encoding a CREG protein or an active fragment thereof in blood, tissue or cells from a subject, wherein an expression level of a CREG protein or an active fragment thereof lower than a reference value is indicative of therapeutic effect and prognosis of overweight, obesity, insulin resistance, or hyperlipidemia.

4. A method of screening a medicament for activity in prevention and/or treatment of overweight, obesity, insulin resistance, hyperlipidemia and related diseases thereof, comprising screening the medicament for up-regulation of expression of CREG protein or an active fragment thereof.

5. (canceled)

6. (canceled)

7. A method for preventing and/or treating overweight, obesity, insulin resistance, hyperlipidemia and related diseases thereof in a subject in need thereof, comprising administering to the subject in need thereof a CREG protein or an active fragment thereof, a nucleic acid molecule encoding a CREG protein or an active fragment thereof, or a recombinant vector or recombinant cell expressing a CREG protein or an active fragment thereof.

8. The method of claim 3, wherein the expression level of the CREG protein or an active fragment thereof is determined by Western blot, or the expression level of the CREG protein is detected by amplification of CREG mRNA by polymerase chain reaction and quantitative reaction.

9. The method of claim 7, wherein administrating reduces body weight without increasing appetite or calorie intake in the subject.

10. The method of claim 7, wherein administrating reduces the degree of hepatic steatosis in the subject.

11. The method of claim 7, wherein administrating reduces plasma total cholesterol, triglyceride and low-density lipoprotein cholesterol levels in the subject.

12. The method of claim 7, wherein administrating improves impaired glucose tolerance and insulin tolerance associated with obesity.

13. The method of claim 7, wherein the disease is metabolic syndrome, pre-diabetes, type 2 diabetes, or dyslipidemia.


SEQUENCE LISTING (2 sequences)

SEQ ID NO: 1 - protein, 220 aa, Homo sapiens
MAGLSRGSARALLAALLASTLLALLVSPARGRGGRDHGDWDEASRLPPLPPREDAARVARFVTHVSDWGALATISTLEAVRGRPFADVLSLSDGPPGAGSGVPYFYLSPLQLSVSNLQENPYATLTMTLAQTNFCKKHGFDPQSPLCVHIMLSGTVTKVNETEMDIAKHSLFIRHPEMKTWPSSHNWFFAKLNITNIWVLDYFGGPKIVTPEEYYNVTVQ

SEQ ID NO: 2 - DNA, 2048 bp, Homo sapiens
ggcggggcctgggcgcgccgagctccggctgggtccctgcaggtcttggggcccgggactcttcctggagacaccgccatggccgggctatcccgcgggtccgcgcgcgcactgctcgccgccctgctggcgtcgacgctgttggcgctgctcgtgtcgcccgcgcggggtcgcggcggccgggaccacggggactgggacgaggcctcccggctgccgccgctaccaccccgcgaggacgcggcgcgcgtggcccgcttcgtgacgcacgtctccgactggggcgctctggccaccatctccacgctggaggcggtgcgcggccggcccttcgccgacgtcctctcgctcagcgacgggcccccgggcgcgggcagcggcgtgccctatttctacctgagcccgctgcagctctccgtgagcaacctgcaggagaatccatatgctacactgaccatgactttggcacagaccaacttctgcaagaaacatggatttgatccacaaagtcccctttgtgttcacataatgctgtcaggaactgtgaccaaggtgaatgaaacagaaatggatattgcaaagcattcgttattcattcgacaccctgagatgaaaacctggccttccagccataattggttctttgctaagttgaatataaccaatatctgggtcctggactactttggtggaccaaaaatcgtgacaccagaagaatattataatgtcacagttcagtgaagcagactgtggtgaatttagcaacacttatgaagtttcttaaagtggctcatacacacttaaaaggcttaatgtttctctggaaagcgtcccagaatattagccagttttctgtcacatgctggtttgtttgcttgcttgtttacttgcttgtttaccaatagagttgacctgttattggatttcctggaagatgtggtagctacttttttcctattttgaagccattttcgtagagaaatatccttcactataatcaaataagttttgtcccatcaattccaaagatgtttccagtggtgctcttgaagaggaatgagtaccagttttaaattgcccattggcatttgaaggtagttgagtatgtgttctttattcctagaagccactgtgcttggtagagtgcatcactcaccacagctgcctcctgagctgcctgagcctggtgcaaaaggattggcccccattatggtgcttctgaataaatcttgccaagatagacaaacaatgatgaaactcagatggagcttcctactcacgttgatttatgtctcacaatcctgggtattgttaattcaacatagggtgaaactatttctgataaagaacttttgaaaaactttttatactctaaagtgatactcagaacaaaagaaagtcataaaactcctgaatttaatttccccacctaagtcgaaacagtattatcaaaacacatgtgcacacagattattttttggctccaaaactggattgcaaaagaaagaggagaagaatattttgtgtgttcctggtattcttttataagtaaagtttacccaggcatggaccagcttcagccagggacaaaatcccctcccaaaccactctccacagctttttaaaaatacttctactcttaacaattacctaaggcttcctcaactgccccaaatctcttaatagcttctagtgctgctacaatctaagtcaggtcaccagagggaagagaacatggcattaaaagaatcacatcttcagaagagaagacactaatattattacccatatacatgatttcagaagatgacataagattcctcttaaagaggaaatgtcaggaatcaagccactgaatccttaaagagaaaagttgaatatgagtcattgtgtctgaaaactgcaaagtgaacttaactgagatccagcaaacaggttctgtttaagaaaaataatttatactaaatttagtaaaatggacttcttattcaaagcatcaataattaaaagaattattttaatgaaaaaaaaaaaaaaaaaaaaaaaaa